(19) United States
(12) Patent Application Publication
(10) Pub. No.: US 2008/0051364 A1
(43) Pub. Date: Feb. 28, 2008

(54) Therapeutic Treatment of Accelerated Bone Resorption

(76) Inventors: Pninna Fishman, Herzliya (IL); Sara Bar Yehuda, Rishon Le Zion (IL); Lea Madi, Rishon Le Zion (IL)

Correspondence Address: BROWDY AND NEIMARK, P.L.L.C.;624 NINTH STREET, NW, SUITE 300, WASHINGTON, DC, 20001-5303, US

(21) Appl. No.: 10/564,620

(22) PCT Filed: Nov. 8, 2005
(86) PCT No.: PCT/IL05/01166

§ 371 Date: Jan. 13, 2006

Related U.S. Application Data
(60) Provisional application No. 60/625,564, filed on Nov. 8, 2004.

(51) Int. Cl. A61K 31/52; A61K 31/70; A61K 47/00; A61P 29/00

(52) U.S. Cl. 514/45; 514/263.1; 514/789

(57) ABSTRACT

The present invention concerns the use of an A₃ adenosine receptor agonist (A₃AR agonist) for treatment of accelerated bone resorption, particularly, inflammation induced bone resorption. Specifically, there is provided by the present invention a method and pharmaceutical composition for treatment of said condition, the A₃AR agonist being formulated as a pharmaceutical composition which is administered to a subject having accelerated bone resorption.

The invention also provides the use of A₃AR agonist in the preparation of said pharmaceutical composition.

### FIELD OF THE INVENTION

This invention relates to therapeutic methods for treatment or prevention of accelerated bone loss.

### PRIOR ART

The following is a list of prior art, which is considered to be pertinent for describing the state of the art in the field of the invention.
(1) Olah M. E. and Stiles G. L. The role of receptor structure in determining adenosine receptor activity, Pharmacol. There., 85:55-75 (2000);
(2) Poulsen S. A. and Quinn R. J., Adenosine receptors: new opportunities for future drugs. Bioorg. Med. Chem., 6:619-641 (1998);
(3) Fang X. et al. Phosphorylation and inactivation of glycogen synthase kinase 3 by protein kinase A., Proc. Natl. Acad. Sci. USA, 97:11960-11965 (2000);
(4) Fishman, P., et al., Involvement of Wet Signaling Pathway in IB-MECA Mediated Suppression of Melanoma Cells, Oncogene 21:4060-4064 (2002);
(5) Ferkey, D. M., and Kimelman, D. GSK-3: New Thoughts on an Old Enzyme, Dev. Biol., 225:471-479 (2000);
(6) Bonvini, P., et al. Nuclear beta-catenin displays GSK-3beta- and APC-independent proteasome sensitivity in melanoma cells, Biochim. Biophys. Acta., 1495:308-318 (2000);
(7) Olah, M. E. and Stiles, G L, The role of receptor structure in determining adenosine receptor activity, Pharmacol. Ther., 85:55-75 (2000);
(8) Szabo C., et al. Suppression of macrophage inflammatory protein (MIP)-1α producing and collagen induced arthritis by adenosine receptor agonists., British Journal of Pharmacology, 125:379-387 (1998);
(9) U.S. Pat. No. 5,773,423;
(10) Nicole C. Walsh and Ellen M. Gravallese. Bone loss in inflammatory arthritis: Mechanism and treatment strategies. Current Opinion in Rheumatology, 16:419-427 (2004);
(11) Zang Hee Lee and Hong-Hee Kim. Signal transduction by receptor activator of nuclear factor kappa B in osteoclasts., Biochemical and Biophysical Research Communication 305:211-214 (2003);

### BACKGROUND OF THE INVENTION

A variety of disorders in humans and other mammals involve or are associated with accelerated bone resorption. Such disorders include, but are not limited to, osteoporosis, Paget's disease, peri-prosthetic bone loss or osteolysis, and hypercalcemia of malignancy. The most common of these disorders is osteoporosis, which in its most frequent manifestation occurs in postmenopausal women. Because the disorders associated with bone loss are chronic conditions, it is believed that appropriate therapy will generally require chronic treatment.

Rheumatoid arthritis (RA) is one example of a chronic inflammatory autoimmune disease which is associated with bone loss. RA affects 1% of the adult population and is characterized by hyperplasia of stromal cells and a massive infiltration of hematopoietic cells into the joints, leading to chronic synovitis and destruction of cartilage, bone, tendons and ligaments. Patients with RA show a reduced bone volume and decreased bone turnover, which is further developed to osteoporosis [Perez-Edo L, et al. J. Scand J Rheumatol., 31:285-290 (2002)]. This progressive joint damage results in functional decline and disability [Harris E D. N. Eng.l J. Med., 322:1277-1289 (1990)]. About 80% of the affected population becomes disabled within 20 years of symptom onset [Paulos C M, et al. Adv. Drug. Deliv. Rev., 56:1205-1217 (2004)].

It is well documented that the bone destruction in RA as well as in other diseases associated with accelerated bone resorption is mainly mediated by osteoclasts and that a member of the TNF family, the receptor activator of NF-κB ligand (RANKL), is required for the differentiation of osteoclasts from their precursor cells and activation of osteoclastogenesis in inflammatory sites as well as promoting osteoclasts' activity and survival [Hsu H, et al. Proc. Natl. Acad. Sci. U.S.A., 96:3540-3545 (1999)]. RANKL is highly expressed on outer plasma membrane of osteoblasts, stromal cells, synovial fibroblasts and T cells in arthritic joints [Kwan Tat S, et al. Cytokine Growth. Factor Rev., 5:49-60 (2004); Kotake S, et al. Arthritis. Rheum., 44:1003-1012 (2001)]. It binds to its receptor RANK, which is present on the osteoclasts progenitors, evoking downstream PI3K-PKB signaling pathway, leading to the activation of the transcription factor NF-κB [Udagawa N, et al. Arthritis. Res., 4:281-289. (2002); Gingery A, et al. J. Cell. Biochem., 89:165-179 (2003)].

Accumulative evidence pointed out that adenosine plays an important role in limiting inflammation, mainly by prevention pro-inflammatory cytokine production such as TNF-α, IL-1 and IL-6 [Cronstein, B. N. J. Appl. Physiol. 76:5-13 (1994); Eigler, A., et al. Scand. J. Immunol., 45:132-139 (1997); Mabley, J., et al. Eur. J. Pharmacol. 466:323-329 (2003)]. Adenosine, which is released into the extra cellular environment from activated or metabolically stimulated cells, binds to selective G-protein-associated membrane receptors, designated A₁, A_{2A}, A_{2B}, and A₃ [Stiles, G. L., Clin. Res. 38:10-18 (1990)]. The anti-inflammatory effect of adenosine was found to be mediated via the A₃AR [Szabo, C., et al. Br. J. Pharmacol. 125:379-387 (1998)]. Specifically, it was shown that the highly selective A₃AR agonist, IB-MECA is efficacious in preventing the clinical and pathological manifestations of arthritis in different experimental animal models which included Adjuvant Induced Arthritis (AIA), collagen induced arthritis (CIA) and thropomyosine induced arthritis. The mechanism of action entailed down-regulation of NF-κB, TNF-α and MIP-1α [Baharav E., et al. J. Rhematol. Accepted (2004)].

### SUMMARY OF THE INVENTION

The present invention is based on the surprising finding that the highly selective A₃AR agonist, IB-MECA, prevents bone loss in an Adjuvant Induced Arthritis (AIA) rat model. As exemplified hereinbelow, this selective agonist down-regulated key signaling proteins such as NF-KB and RANKL resulting in down-regulation of TNF-α, leading to the prevention of bone loss.

Thus, according to a first aspect, the present invention provides a method for the treatment of accelerated bone resorption in a mammal subject comprising administering to said subject in need of said treatment an amount of an A₃ adenosine receptor agonist (A₃AR agonist), the amount being effective to inhibit bone resorption.

The term “treatment” as used herein denotes curative as well as prophylactic treatment. Specifically, treatment includes inhibition of accelerated bone resorption and of the development of osteolytic lesions. Without being limited thereto, treatment of bone resorption encompass amelioration of undesired symptoms associated with bone resorption (e.g. pain, bone fractures, spinal cord compression, and hypercalcemia), prevention of the manifestation of such symptoms before they occur, slowing down or prevention of irreversible damage caused by chronic stages of a disease associated with bone loss (e.g. preventing the development of osteolytic lesions and fractions), lessening the severity of diseases associated with bone resorption, improvement of bone recovery, prevention of bone resorption from developing, prevention of bone tissue death, as well as any improvement in the well being of the patients. For example, an improvement may be manifested by one or more of the following: increase in bone mass, relief of pain associated with bone resorption, reduction in bone fractioning and others. According to the invention, treatment may also include a combination of two or more of the above.

The term “accelerated bone resorption” which may be used interchangeably with the terms “accelerated bone loss”, “accelerated bone destruction” and “Osteoclastic bone” in the context of the present invention refers to any disease, disorder or pathological condition which involves the development of osteoclastic bone and may be either as a result of a metabolic bone disease, from accelerated metabolic processes induced by inflammation or by any other pathological condition. Non-limiting examples of diseases involved with bone resorption include osteoporosis, Paget's disease, peri-prosthetic bone loss, osteonecrosis (death or destruction of bone tissue due to trauma, loss of blood supply or disease), myeloma bone disease, osteolysis, and hypercalcemia of malignancy.

The term “A₃ adenosine receptor agonist” (A₃AR agonist) in the context of the present invention refers to any compound capable of specifically binding to the A₃ adenosine receptor (“A₃AR”), thereby fully or partially activating said receptor. The A₃AR agonist is thus a compound that exerts its prime effect through the binding and activation of the A₃AR. Preferred embodiments of A₃AR agonists are provided hereinafter.

The “amount” (herein also termed the “effective amount”) of A₃AR agonist in the context of the present invention refers to an amount effective to provide protection of a mammal from bone resorption as well as from the development of diseases associated with bone resorption. An amount being effective to provide the desired protection can be readily determined, in accordance with the invention, by administering to a plurality of tested subjects various amounts of the A₃AR agonist and then plotting the physiological response (for example an integrated “SS index” combining several of the therapeutically beneficial effects) as a function of the amount. Alternatively, the effective amount may also be determined, at times, through experiments performed in appropriate animal models and then extrapolating to human beings using one of a plurality of conversion methods; or by measuring the plasma concentration or the area under the curve (AUC) of the plasma concentration over time and calculating the effective dose so as to yield a comparable plasma concentration or AUC. As known, the effective amount may depend on a variety of factors such as mode of administration (for example, oral administration may require a higher dose to achieve a given plasma level or AUC than an intravenous administration); the age, weight, body surface area, gender, health condition and genetic factors of the subject; other administered drugs; etc.

In the following, unless otherwise indicated, dosages are indicated in weight/Kg, meaning weight of administered A₃AR agonist per kilogram of body weight of the treated subject in each administration. For example, mg/Kg and microgram/Kg denote, respectively, milligrams of administered agent and micrograms of administered agent per kilogram of body weight of the treated subject.

In mice the effective amount is typically less than about 1000 and preferably less than about 500 microgram/Kg. A typical dose would be in the range of about 1 microgram/Kg to about 200 microgram/Kg, with a preferred dose being in the range of about 5 microgram/Kg to about 150 microgram/Kg. The corresponding effective amount in a human will be a human equivalent amount to that observed in mice, which may be determined in a manner as explained bellow.

The term “human equivalent” refers to the dose that produces in human the same effect as featured when a dose of 0.001-1 mg/Kg of an A₃AR agonist is administered to a mouse or a rat. As known, this dose depends and may be determined on the basis of a number of parameters such as body mass, body surface area, absorption rate of the active agent, clearance rate of the agent, rate of metabolism and others.

The human equivalent may be calculated based on a number of conversion criteria as explained bellow; or may be a dose such that either the plasma level will be similar to that in a mouse following administration at a dose as specified above; or a dose that yields a total exposure (namely area under the curve, ‘AUC’, of the plasma level of said agent as a function of time) that is similar to that in mice at the specified dose range.

It is well known that an amount of X mg/Kg administered to rats can be converted to an equivalent amount in another species (notably humans) by the use of one of possible conversions equations well known in the art. Examples of conversion equations are as follows:

### Conversion I:

| Species | Body Wt. (Kg) | Body Surf. Area (m²) | Km Factor |
|---|---|---|---|
| Mouse | 0.2 | 0.0066 | 3.0 |
| Rat | 0.15 | 0.025 | 5.9 |
| Human Child | 20.0 | 0.80 | 25 |
| Adult | 70.0 | 1.60 | 37 |

Body Surface area dependent Dose conversion: Rat (150 g) to Man (70 Kg) is 1/7 the rat dose. This means that, for example, 0.001-1 mg/Kg in rats equals to about 0.14-140 microgram/Kg in humans. Assuming an average human weight of 70 Kg, this would translate into an absolute dosage for humans of about 0.01 to about 10 mg.

### Conversion II:

The following conversion factors: Mouse=3, Rat=67. Multiply the conversion factor by the animal weight to go from mg/Kg to mg/m² for human dose equivalent.

| Species | Weight (Kg) | BSA (m²) |
|---|---|---|
| Human | 70.00 | 1.710 |
| Mouse | 0.02 | 0.007 |
| Rat | 0.15 | 0.025 |
| Dog | 8.00 | 0.448 |

According to this equation the amounts equivalent to 0.001-1 mg/Kg in rats for humans are 0.16-64 μg/Kg; namely an absolute dose for a human weighing about 70 Kg of about 0.011 to about 11 mg, similar to the range indicated in Conversion I.

### Conversion III:

Another alternative for conversion is by setting the dose to yield the same plasma level or AUC as that achieved following administration to an animal. For example, based on measurement made in mice following oral administration of IB-MECA and based on such measurements made in humans in a clinical study in which IB-MECA was given to healthy male volunteers it can be concluded that a dose of 1 microgram/Kg-1,000 microgram/KG in mice is equivalent to a human dose of about 0.14-140 microgram/Kg, namely a total dose for a 70 Kg individual of 0.01-10 mg.

Based on the above conversion methods, the preferred dosage range for two specific A₃AR agonist, e.g. IB-MECA and Cl-IB-MECA, would be less than 4 mg, typically within the range of about 0.01 to about 2 mg (about 0.14-28 micrograms/Kg, respectively) and desirably within the range of about 0.1 to 1.5 mg (about 1.4-21 micrograms/Kg, respectively). This dose may be administered once, twice or, at times, several times a day. Human studies showed (data not shown herein) that the level of IB-MECA decays in the human plasma with a half life of about 8-10 hours, as compared to a half life of only 1.5 hours in mice, in case of multiple daily administration, correction in the dosages for accumulative effects needs to be made at times (a subsequent dose is administered before the level of a previous one was decayed and thus there is a build-up of plasma level over that which occurs in a single dose. On the basis of said human trials once or twice daily administration appears to be a preferred administration regiment. However this does not rule out other administration regiments.

It should be noted that in addition to said therapeutic method, also encompassed within the present invention is a pharmaceutical composition for the treatment of accelerated bone resorption, the composition comprising as the active ingredient an amount of an A₃AR agonist and a pharmaceutically acceptable carrier, the amount being effective to inhibit bone resorption in a subject in need of said treatment.

The term “pharmaceutically acceptable carrier” in the context of the present invention denotes any one of inert, non-toxic materials, which do not react with the A₃AR agonist and which can be added to formulations as diluents, carriers or to give form or consistency to the formulation.

The carrier also includes substances for providing the formulation with stability, sterility and isotonicity (e.g. antimicrobial preservatives, antioxidants, chelating agents and buffers), for preventing the action of microorganisms (e.g. antimicrobial and antifungal agents, such as parabens, chlorobutanol, phenol, sorbic acid and the like), for providing the formulation with an edible flavor or with a color etc.

The carrier may also at times have the effect of the improving the delivery or penetration of the A₃AR agonist to the target tissue, for improving the stability of the A₃AR agonist, for slowing clearance rates, for imparting slow release properties, for reducing undesired side effects etc.

Further, the present invention encompasses the use of an A₃AR agonist for the preparation of a pharmaceutical composition for the treatment of accelerated bone resorption.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIGS. 1A-1C include graphs showing the clinical score (FIG. 1A) and paw thickness (FIG. 1B) after treatment with IB-MECA of AIA rats, as well as pictures (FIG. 1C) demonstrating the severe redness and swelling of the entire paw in the control group (left picture), in comparison to a representative paw in the IB-MECA treated group, which appears completely normal (right picture).

FIGS. 2A-2C include a bar graph providing inflammation score (FIG. 2A) as well as histological cross sections (x20 and x40) (FIG. 2B) showing the change in inflammation in the joints of IB-MECA treated rates compared to control rats.

FIGS. 3A-3B include a bar graph of fibrosis score (FIG. 3A) as well as istological cross sections (FIG. 3B), showing the change in the synovium in IB-MECA treated rates, compared to control rats.

FIGS. 4A-4B include a bar graph of pannus score (FIG. 4A) and histological cross sections (FIG. 4B) showing the change in the pannus tissue in the articular space of IB-MECA treated rats, compared to control rats

FIGS. 5A-5B include a bar graph of cartilage destruction score (FIG. 5A) as well as histological cross sections (FIG. 5B) showing the change in the cartilage in IB-MECA treated rats, compared to control rats.

FIGS. 6A-6B include a bar graph of osteoclasts score (FIG. 6A) as well as histological cross sections (FIG. 6B) showing the change in the appearance of osteoclasts in IB-MECA treated rats, comparted to the control rats.

FIGS. 7A-7B include a bar graph of bone destruction score (FIG. 7A) as well as histological cross sections (FIG. 7B) showing the change in bone mass in IB-MECA treated rats, compared to the control rats.

FIGS. 8A-8B include a bar graph of osteoblasts score (FIG. 8A) as well as histological cross sections (FIG. 8B) showing the change in osteoblasts population in IB-MECA treated rats, compared to the control rats.

FIGS. 9A-9B include a bar graph of new bone formation score (FIG. 9A) as well as histological cross sections (FIG. 9B) showing new bone formation in IB-MECA treated rats, compared to untreated group.

FIGS. 10A-10D show the effect of IB-MECA treatment on the expression of A3AR (FIG. 10A) and additional key regulatory proteins in paw extracts, including RANKL (FIG. 10B), PI3K; PKB/Akt; IKKα,β; NF-κB and TNF-α (FIG. 10C) as well as white blood (WB) analysis of the apoptotic enzyme caspase-3 (FIG. 10D).

### DETAILED DESCRIPTION OF THE INVENTION

The process of bone formation (osteogenesis) involves three main steps: production of production of the extracellular organic matrix (osteoid); mineralization of the matrix to form bone; and bone remodeling by resorption and reformation. The cellular activities of osteoblasts, osteocytes, and osteoclasts are essential to the process. Osteoblasts synthesize the collagenous precursors of bone matrix and also regulate its mineralization. As the process of bone formation progresses, the osteoblasts come to lie in tiny spaces (lacunae) within the surrounding mineralized matrix and are then called osteocytes. To meet the requirements of skeletal growth and mechanical function, bone undergoes dynamic remodeling by a coupled process of bone resorption by osteoclasts and reformation by osteoblasts.

Several metabolic bone diseases (such as hyperparathyroidism, Paget's disease, and others) are characterized by increased modeling and increased osteoclastic activity. In addition, osteoclasts and osteoclastlike cells have been identified as important effector cells in mediating inflammation-induced bone loss in, for example, inflammatory arthritis (e.g. rheumatoid arthritis and psoriatic arthritis).

The present invention aims for the providence of a method for inhibiting accelerated bone resorption and thereby curing or preventing the consequences of abnormal bone loss. Thus, according to a first aspect there is provided a method for the treatment of accelerated bone resorption in a mammal subject, the method comprises administering to said subject in need of said treatment an amount of an A₃ adenosine receptor agonist (A₃AR agonist), the amount being effective to inhibit bone resorption.

As defined hereinbefore, A₃AR agonist is preferably a compound that exerts its prime effect through the binding and activation of the A₃AR. In one embodiment, an A₃AR agonist has a binding affinity (Kᵢ) to the human adenosine A₃ receptor in the range of less than 100 nM, typically less than 50 nM, preferably less than 20 nM, preferably less than 10 nM and ideally less than 5 nM. According to this embodiment, preferred are A₃RAgs that have a Kᵢ to the human A₃R of less than 2 nM and desirably less than 1 nM.

It should be noted that some A₃AR agonists can also interact with and activate other receptors with lower affinities (namely a higher Ki). A compound will be considered an A₃AR agonist in the context of the invention (namely a compound that exerts its prime effect through the binding and activation A₃AR) if its affinity to the A₃AR is at least 3 times (i.e. its Ki to the A₃AR is at least 3 times lower), preferably 10 times, desirably 20 times and most preferably at least 50 times larger than the affinity to any other of the adenosine receptors (i.e. A₁, A₂ₐ and A_{2b}).

The affinity of an A₃AR agonist to the human A₃AR as well as its relative affinity to the other human adenosine receptors (A₁, A₂ₐ and A_{2b}) can be determined by a number of assays, such as a binding assay. Examples of binding assays include providing membranes containing a receptor and measuring the ability of the A₃AR agonist to displace a bound radioactive agonist; utilizing cells that display the respective human adenosine receptor and measuring, in a functional assay, the ability of the A₃AR agonist to activate or deactivate, as the case may be, downstream signaling events such as the effect on adenylate cyclase measured through increase or decrease of the cAMP level; etc. Clearly, if the administered level of an A₃AR agonist is increased such that its blood level reaches a level approaching that of the Ki of the A₁, A₂ₐ and A_{2b} adenosine receptors, activation of these receptors may occur following such administration, in addition to activation of the A₃AR. An A₃AR agonist is thus preferably administered at a dose such that the blood level is such so that essentially only the A₃AR will be activated.

The characteristic of some A₃AR agonists and methods of their preparation are described in detail in, inter alia, U.S. Pat. No. 5,688,774; U.S. Pat. No. 5,773,423, U.S. Pat. No. 5,573,772, U.S. Pat. No. 5,443,836, U.S. Pat. No. 6,048,865, WO 95/02604, WO 99/20284, WO 99/06053, WO 97/27173 and applicant's co-pending patent application Ser. No. 09/700,751 (corresponding to WO01/19360), all of which are incorporated herein by reference.

According to one embodiment of the invention, the A₃AR agonist is a compound that exerts its prime effect through the binding and activation A₃AR and is a purine derivative falling within the scope of the general formula (I) and physiologically acceptable salts of said compound:

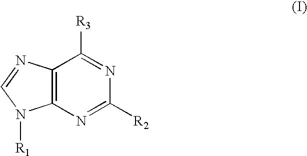

wherein,

R₁ represents an alkyl, hydroxyalkyl, carboxyalkyl or cyanoalkyl or a group of the following general formula (II):

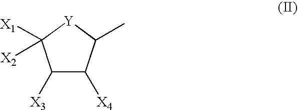

in which:

Y represents oxygen, sulfur or CH₂;

X₁ represents H, alkyl, R^{a}R^{b}NC(═O)— or HOR^{c}—, wherein
R^{a} and R^{b} may be the same or different and are selected from the group consisting of hydrogen, allyl, amino, haloalkyl, aminoalkyl, BOC-aminoalkyl, and cycloalkyl or are joined together to form a heterocyclic ring containing two to five carbon atoms; and
R^{c} is selected from the group consisting of alkyl, amino, haloalkyl, aminoallyl, BOC-aminoalkyl, and cycloalkyl;

X₂ is H, hydroxyl, alkylamino, alkylamido or hydroxyallyl;

X₃ and X₄ represent independently hydrogen, hydroxyl, amino, amido, azido, halo, alkyl, alkoxy, carboxy, nitrilo, nitro, trifluoro, aryl, alkaryl, thio, thioester, thioether, —OCOPh, —OC(═S)OPh or both X₃ and X₄ are oxygens connected to >C═S to form a 5-membered ring, or X₂ and X₃ form the ring of formula (III):

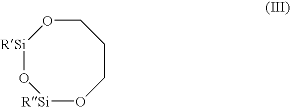

where R′ and R″ represent independently an alkyl group;

R₂ is selected from the group consisting of hydrogen, halo, alkylether, amino, hydrazido, alkylamino, alkoxy, thioalkoxy, pyridylthio, alkenyl; alkynyl, thio, and alkylthio; and

R₃ is a group of the formula —NR₄R₅ wherein

R₄ is a hydrogen atom or a group selected from alkyl, substituted alkyl or aryl-NH—C(Z)-, with Z being O, S, or NR^{a} with R^{a} having the above meanings; wherein when R₄ is hydrogen than

R₅ is selected from the group consisting of R- and S-1-phenylethyl, benzyl, phenylethyl or anilide groups unsubstituted or substituted in one or more positions with a substituent selected from the group consisting of alkyl, amino, halo, haloalkyl, nitro, hydroxyl, acetoamido, alkoxy, and sulfonic acid or a salt thereof; benzodioxanemethyl, fururyl, L-propylalanyl-aminobenzyl, β-alanylaminobenzyl, T-BOC-β-alanylaminobenzyl, phenylamino, carbamoyl, phenoxy or cycloallyl; or R₅ is a group of the following formula:

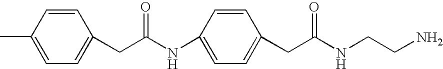

or when R₄ is an alkyl or aryl-NH—C(Z)-, then, R₅ is selected from the group consisting of heteroaryl-NR^{a}-C(Z)-, heteroaryl-C(Z)-, alkaryl-NR^{a}-C(Z)-, alkaryl-C(Z)-, aryl-NR-C(Z)- and aryl-C(Z)-; Z representing an oxygen, sulfor or amine; or a physiologically acceptable salt of the above compound.

According to one preferred embodiment, the A₃RAg is a nucleoside derivative of the general formula (IV):

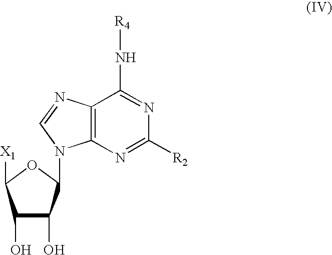

wherein X₁, R₂ and R₄ are as defined above, and physiologically acceptable salts of said compound.

The non-cyclic carbohydrate groups (e.g. alkyl, alkenyl, alkynyl, alkoxy, aralkyl, alkaryl, alkylamine, etc) forming part of the substituent of the compounds of the present invention are either branched or unbranched, preferably containing from one or two to twelve carbon atoms.

The term “alkyl” as used herein denotes any saturated carbohydrate, either linear or branched. The term “lower alkyl” as used herein denotes a saturated carbohydrate (linear or branched) comprising from 1 to about 10 carbon atoms in the backbone.

The terms “alkenyl” and “alkynyl” as used herein denote refer to linear or branched carbohydrates wherein at least two adjacent carbon atoms are connected via a double or triple bond, respectively. Accordingly, the the terms “lower alkenyl” and “lower alkynyl” refer to linear or branched carbohydrates comprising from 2 to 10 carbon atoms in the backbone.

When referring to “physiologically acceptable salts” of the A₃AR agonist employed by the present invention it is meant any non-toxic alkali metal, alkaline earth metal, and ammonium salt commonly used in the pharmaceutical industry, including the sodium, potassium, lithium, calcium, magnesium, barium ammonium and protamine zinc salts, which are prepared by methods known in the art. The term also includes non-toxic acid addition salts, which are generally prepared by reacting the compounds of this invention with a suitable organic or inorganic acid. The resulting acid addition salts are those which retain the biological effectiveness and qualitative properties of the free bases and which are not toxic or otherwise undesirable. Examples include, inter alia, acids derived from mineral acids, hydrochloric, hydrobromic, sulfuric, nitric, phosphoric, metaphosphoric and the like. Organic acids include, inter alia, tartaric, acetic, propionic, citric, malic, malonic, lactic, fumaric, benzoic, cinnamic, mandelic, glycolic, gluconic, pyruvic, succinic salicylic and arylsulphonic, e.g. p-toluenesulphonic, acids.

Specific examples of A₃AR agonist which may be employed according to general formula (IV) of the present invention include, without being limited thereto, N⁶-2-(4-aminophenyl)ethyladenosine (APNEA), N⁶-(4-amino-3-iodobenzyl) adenosine-5′-(N-methyluronamide) (AB-MECA), N⁶-(3-iodobenzyl)-adenosine-5′-N-methyluronamide (IB-MECA) and 2-chloro-N⁶-(3-iodobenzyl)-adenosine-5′-N-methyluronamide (Cl-IB-MECA).

A preferred A₃AR agonist according to the invention is IB-MECA.

Yet, according to another embodiment, the A₃AR agonist may be an oxide derivative of adenosine, such as N⁶-benzyladenosine-5′-N-alkyluronamide-N¹-oxide or N⁶-benzyladenosine-5′-N-dialkyluronamide-N¹-oxide, wherein the 2-purine position may be substituted with an alkoxy, amino, alkenyl, alkynyl or halogen.

Accelerated bone loss may be due to an accelerated metabolic process, as a result of a bone disease, or induced by inflammation. As appreciated by those versed in the art, long-term inflammation can have the effect of removing calcium from the bones, weakening and shrinking them. Inflammation-mediated bone loss occurs in various diseases such as periodontal disease, osteo- and rheumatoid arthritis and some forms of osteoporosis.

Thus, according to one embodiment, the invention concerns treatment of accelerated bone resorption induced by inflammation. According to a preferred embodiment, the method of the invention is for the accelerated bone resorption resulting from inflammatory arthritis.

The invention also concerns pharmaceutical compositions for the treatment of accelerated bone resorption as detailed hereinbefore, the composition comprising as the active ingredient an amount of an A₃AR agonist and a pharmaceutically acceptable carrier, the amount being effective to inhibit bone resorption in a subject in need of said treatment.

The composition of the present invention is administered and dosed in accordance with good medical practice, taking into account the clinical condition of the individual patient, the site and method of administration, scheduling of administration, patient age, sex, body weight and other factors known to medical practitioners. The choice of carrier will be determined in part by the particular active ingredient, as well as by the particular method used to administer the composition. Accordingly, there is a wide variety of suitable pharmaceutical compositions of the present invention.

According to a preferred embodiment, the pharmaceutical composition is in a form suitable for oral administration. Typical examples of carriers suitable for oral administration include (a) liquid solutions, where an effective amount of the A₃AR agonist is dissolved in diluents, such as water, saline, natural juices, alcohols, syrups, etc.; (b) capsules (e.g. the ordinary hard- or soft-shelled gelatin type containing, for example, surfactants, lubricants, and inert fillers), tablets, lozenges (wherein the A₃AR agonist is in a flavor, such as sucrose or the A₃AR agonist is in an inert base, such as gelatin and glycerin), and troches, each containing a predetermined amount of A₃AR agonist as solids or granules; (c) powders; (d) suspensions in an appropriate liquid; (e) suitable emulsions; (f) liposome formulation; and others.

Further, the invention concerns the use of A₃AR agonist for the preparation of a pharmaceutical composition for the treatment of accelerated bone resorption.

The invention will now be exemplified in the following description of experiments that were carried out in accordance with the invention. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways, than as specifically described hereinbelow.

### DESCRIPTION OF SPECIFIC EXAMPLES

### Materials & Methods

### Drugs

The A₃AR agonist, a GMP grade of the compound known generically as 1-Deoxy-1-[6-[[(3-iodophenyl)methyl]amino]-9H-purine-9-yl]-N-methyl-D-ribofuranuronamide or as N⁶-(3-iodobenzyl)-adenosine-5′-N-methyluronamide (IB-MECA), was synthesized for Can-Fite BioPharma by Albany Molecular Research Inc, Albany, N.Y., USA. A stock solution of 10 mM was prepared in DMSO and further dilutions in culture medium or PBS were performed to reach the desired concentration.

Incomplete Freund's adjuvant was purchased from Sigma and heat killed Mycobacterium tuberculosis H37Ra, from Difco (Detroit, USA).

Rabbit polyclonal antibodies against rat A₃AR, and the signaling proteins IKK, TNF-α, GSK-3β, caspase-3 and phospho-specific PKB/Akt, RANKL were purchased from Santa Cruz Biotechnology Inc., Ca, USA. The NF-κB antibody was purchased from cell signaling.

### Animal Models

Experiments were performed in accordance with the guidelines established by the Institutional Animal Care and Use Committee at Can-Fite BioPharma, Kiryat-Matalon, Petach Tikva, Israel. Animals received standardized pelleted diet and tap water ad libitum.

Female Lewis rats, aged 8-10 weeks, obtained from Harlan Laboratories (Jerusalem, Israel), were injected subcutaneously (SC) at the tail base with 100 μl of suspension composed of incomplete Freund's adjuvant with 10 mg/ml heat killed Mycobacterium tuberculosis. Each group contained 10 animals and each experiment was conducted at least three times.

### Treatment Protocols

Drugs were orally administered by gavage, twice daily. The positive control received vehicle only (DMSO in a dilution corresponding to that of the drug) while the treatment groups received 10 μg/kg of IB-MECA. Treatment was initiated on day 14 after vaccination.

### Clinical Disease Score

The animals were inspected every second day for clinical arthritis. The scoring system ranged from 0-4 of each limb: 0—no arthritis; 1—redness or swelling of one toe/finger joint; 2—redness and swelling of more than one toe/finger joints, 3—the ankle and tarsal-metatarsal joints involvement. 4—entire paw redness or swelling. The inflammatory intensity was also determined in accordance with the increase in the rat hind paw's diameter, measured by caliper (Mitotoyo, Tokyo, Japan).

### Histological Score

The foot, knee and hip region of both vehicle and CF101 treated animals were collected and fixed in 10% buffered formalin and decalcified in hydrochloric acid (Calci-Clear Rapid) (Pational Diagnostics, Gr, USA) for 24 h. The specimens were then processed for paraffine embedding, histologic 4-μm sections were cut and stained with hematoxylin and eosin. The sections were assessed by a pathologist blinded to the treatment protocols, and each joint was scored separately. The histology score was assessed as follows: A score of 0 to 4 for the extent of inflammatory cells' infiltration to was used according to the followed: 0—Normal; 1—minimal inflammatory infiltration; 2—mild infiltration; 3—moderate infiltration; 4—marked infiltration. The pannus formation joint tissues, synovial lining cell hyperplasia. The score was graded 0-4: 0—normal; 1—minimal loss of cortical bone at a few sites; 2—mild loss of cortical trabecular bone; 3—moderate loss of bone at many sites; 4—marked loss of bone at many sites; 5—marked loss of bone at many sites with fragmenting and full thickness penetration of inflammatory process or pannus into the cortical bone. The mean of all the histological parameter scores were designated “Histology Score”.

### Protein Extraction from Paw

The hind paws were dissected above the ankle joint. The bony tissue was broken into pieces broken, snap frozen in liquid nitrogen and stored at −80° C. until use. The paw tissues were added to (4 ml/g tissue) RIPA extraction buffer containing 150 mM NaCl, 50 mM Tris, 1% NP40, 0.5% Deoxycholate and 0.1% SDS. Tissues were homogenized on ice with a polytron, centrifuged and the supernatans were subjected to Western Blot analysis

### Western Blot Analysis

Western blot analysis (WB) of paw extracts were carried out according to the following protocol. Samples were rinsed with ice-cold PBS and transferred to ice-cold lysis buffer (TNN buffer, 50 mM Tris buffer pH=7.5, 150 mM NaCl, NP 40). Cell debris was removed by centrifugation for 10 min, at 7500×g. Protein concentrations were determined using the Bio-Rad protein assay dye reagent. Equal amounts of the sample (50 μg) were separated by SDS-PAGE, using 12% polyacrylamide gels. The resolved proteins were then electroblotted onto nitrocellulose membranes (Schleicher & Schuell, Keene, N.H., USA). Membranes were blocked with 1% BSA and incubated with the desired primary antibody (dilution 1:1000) for 24 h at 4° C. Blots were then washed and incubated with a secondary antibody for 1 h at room temperature. Bands were recorded using BCIP/NBT color development kit (Promega, Madison, Wis., USA). Data presented in the different figures are representative of at least four different experiments.

### Results

### Effects of IB-MECA on the Development of Arthritis Score in the AIA Model

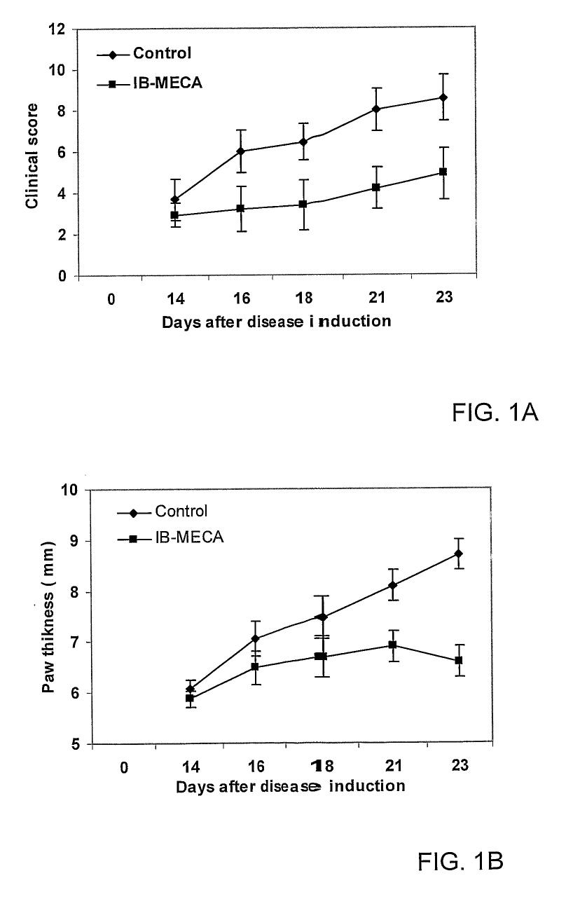

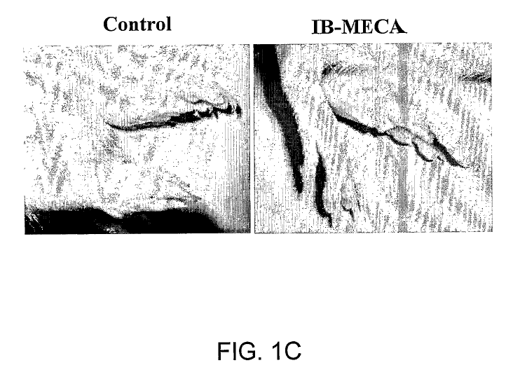

The clinical signs of the arthritis started to appear on day 14. In the control group the maximal clinical arthritic score reached up to 8.7±0.76, while in the IB-MECA treated group the maximal clinical arthritic score had a lower value of 4.8±0.95 (FIG. 1A). The IB-MECA treatment significantly decreased the paw edema. Also, IB-MECA treatment resulted in a 35%±1.2 inhibition in the paw thickness (FIG. 1B). FIG. 1C is a picture demonstrating the severe redness and swelling of the entire paw in the control group, in comparison to a representative paw in the IB-MECA treated group, which appears completely normal (right).

### Effects of IB-MECA on the Histological Features of AIA

At day 23 after the disease induction, the animals were scarified, and joints from two hind paws of each animal were harvested and examined histologically. The histological analysis was carried out on the basis of infiltration of inflammatory cells, synovial hyperplasia, cartilage and bone destruction. Most of the histophathological changes were found in the interophalangeal region of the foot. Similar changes in the knee region were noted in the vehicle group, while the knee in the IB-MECA treated group remained intact, demonstrating the severity of the disease in the untreated group. Overall, the severity of joint histopathology was correlated with the clinical severity index.

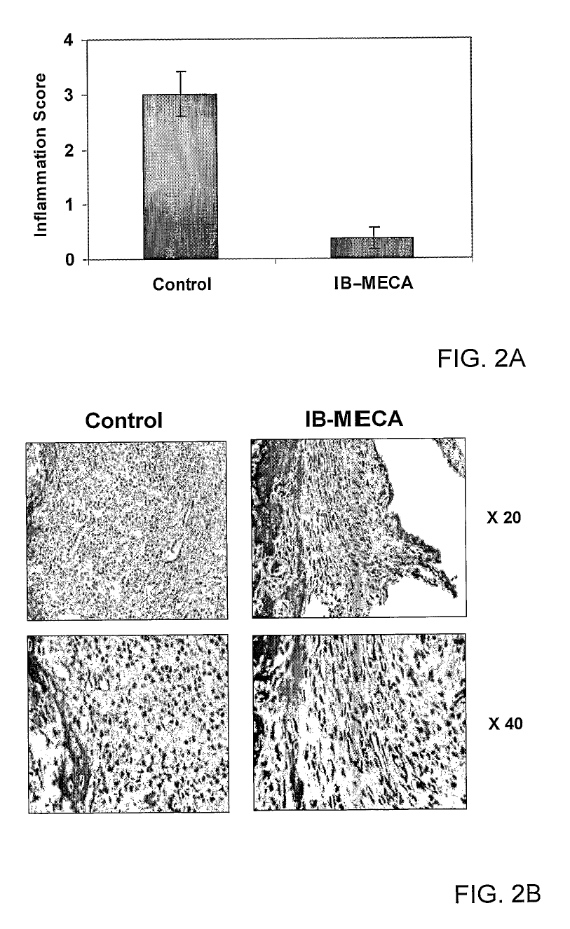

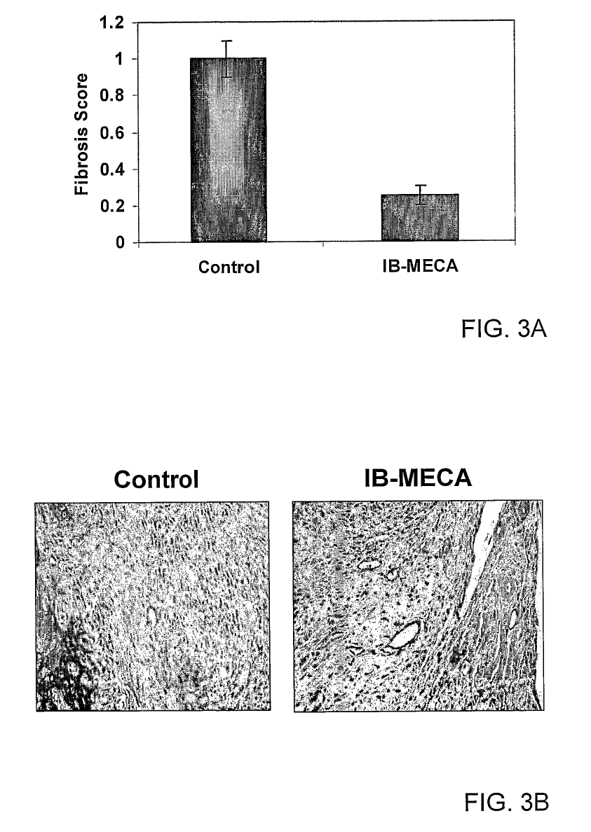

A statistically significant reduction in inflammatory changes was seen in the joints of treated rats compared to control rats control group in which extensive area of inflammation was noted (mean total score 0.4±0.034 vs. 3.2±0.14, respectively) (FIGS. 2A-2B). The synovium appeared thickened, fibrous, hyperplastic and hypertrophic due to resident synovial cell proliferation and infiltration by mononuclear leukocytes was noted in the control group (FIGS. 3A-3B). On the contrary, almost no the fibrosis and mild hyperplasia of the synovia was observed in the IB-MECA treated group.

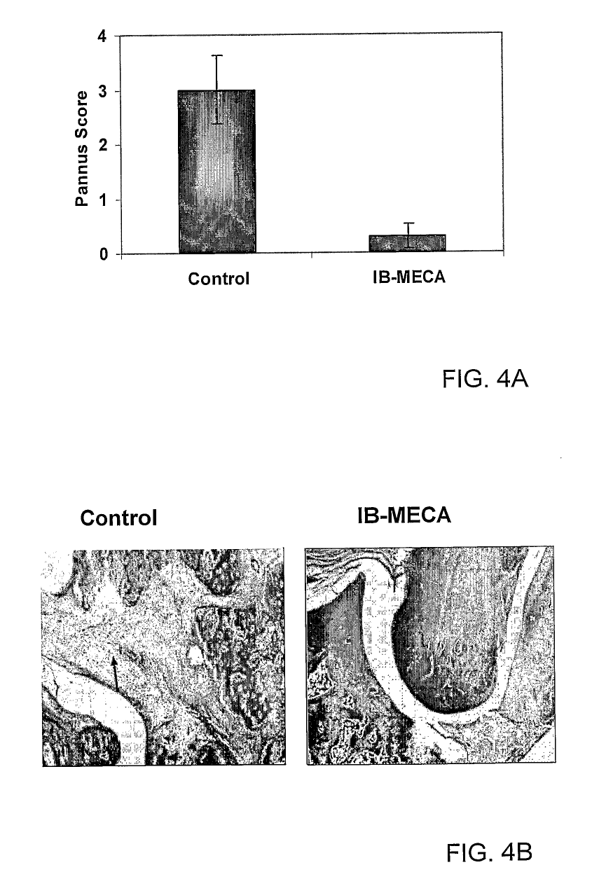

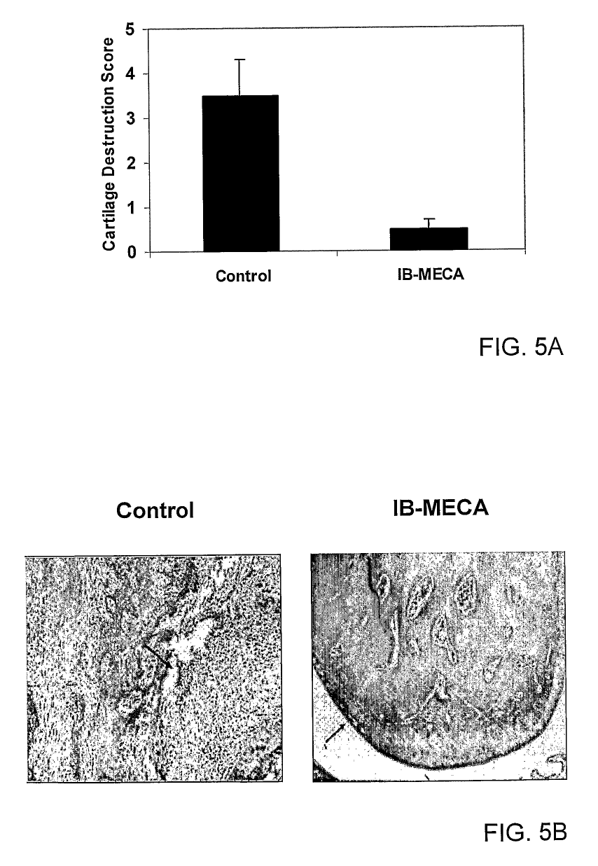

In the control group massive pannus tissue was present in the destructed areas replacing the normal tissue of the articular space, while in the IB-MECA treated group there was a mild evidence for a development of pannus tissue (FIGS. 4A-4B). Severe cartilage damage followed by cartilage loss was presented in the control group, whereas in the IB-MECA treated animals the cartilage texture appeared to be normal (FIGS. 5A-5B).

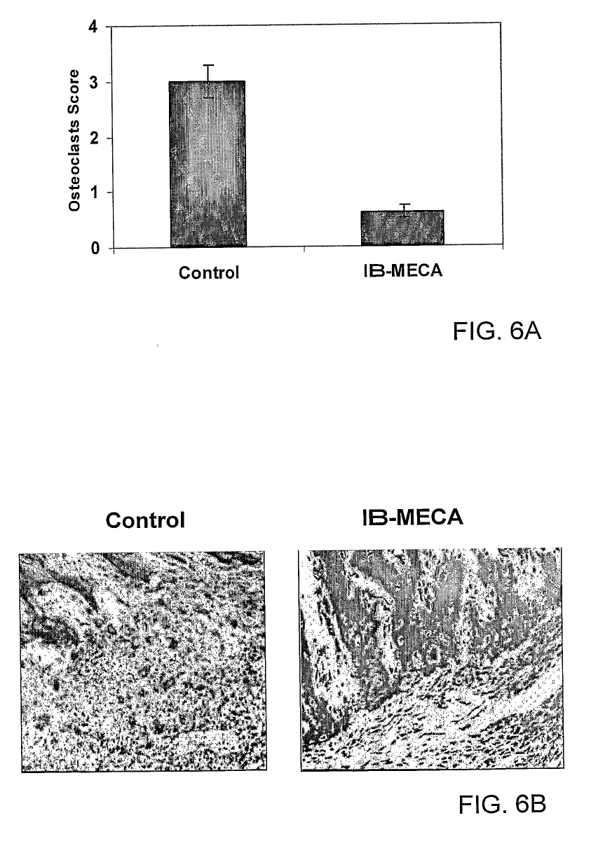

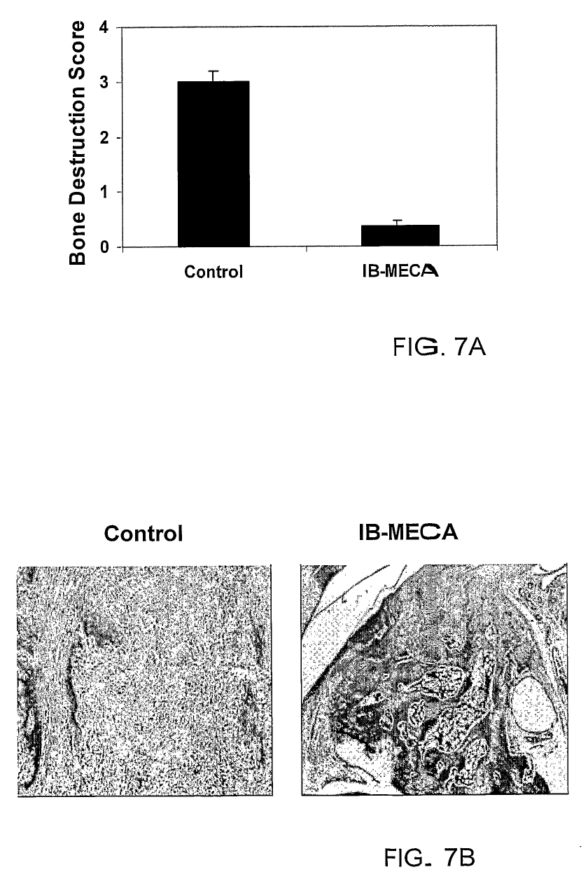

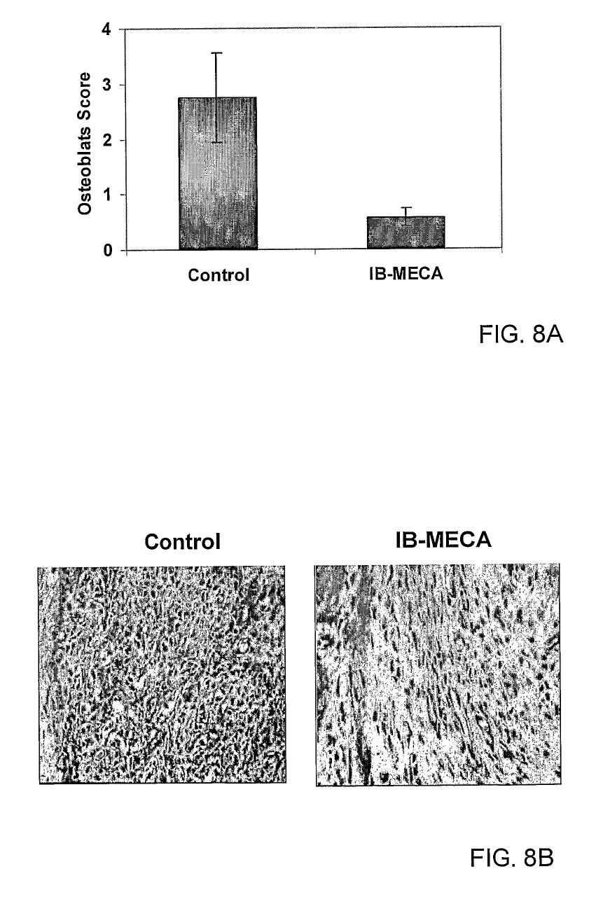

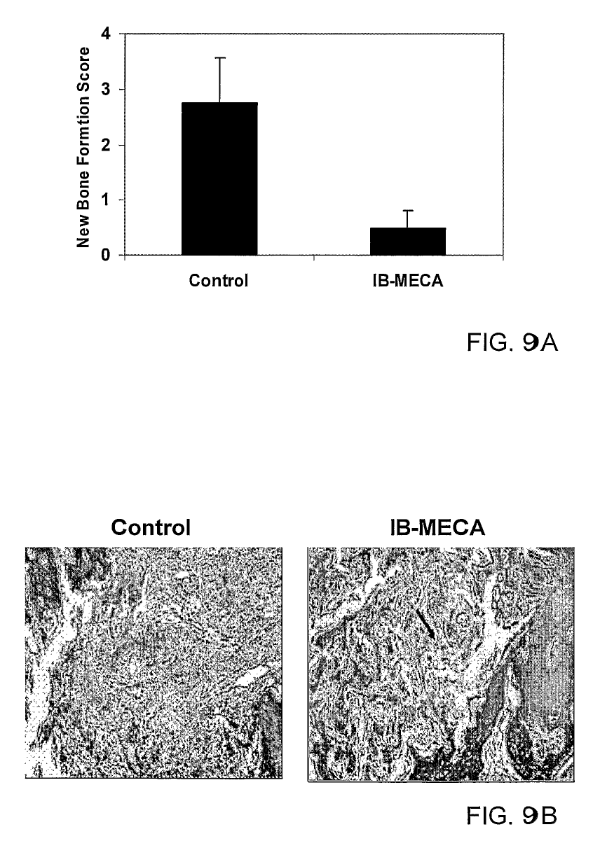

A reduction of 73% in the appearance of osteoclasts was note in the IB-MECA treated group in comparison to the control group (FIGS. 6A-6B). This was followed by a significant high level of bone destraction in the control group (FIGS. 7A-7B). Consequently, a low presence of osteoblasts (FIGS. 8A-8B) and slight new bone formation was observed in the IB-MECA treated animals (FIGS. 9A-9B).

### Effects of IB-MECA on the Level of Key Signaling Protein Expression Down-Stream to A3AR Activation in Paw Extracts Derived from AIA Rats

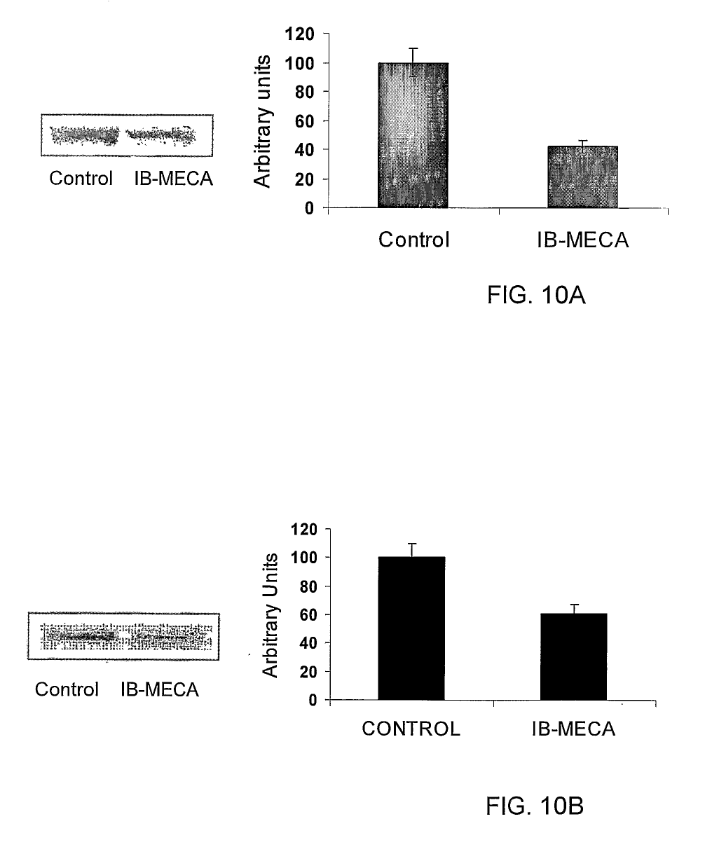

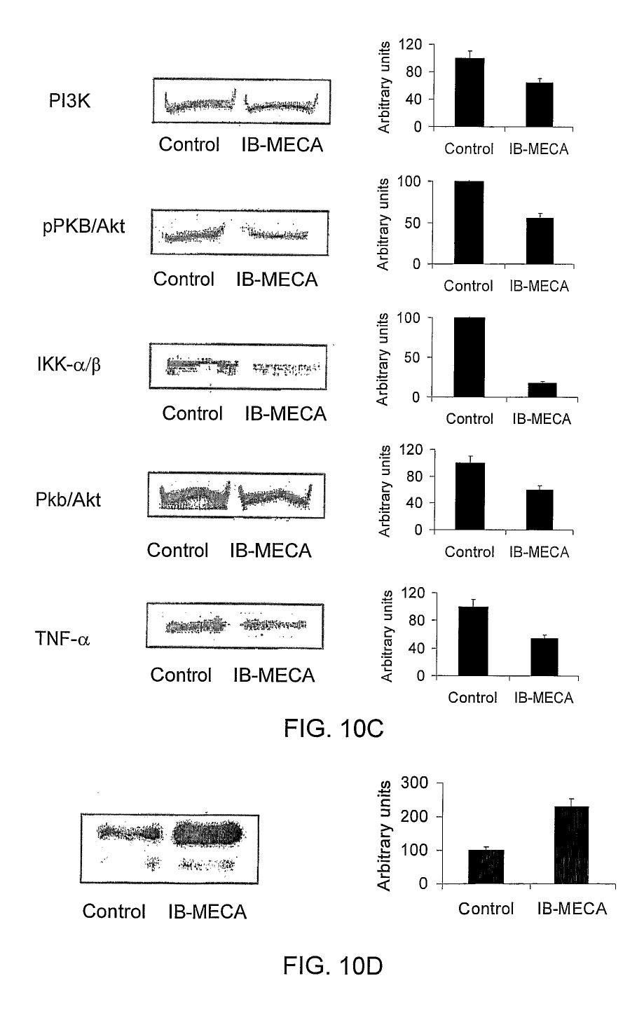

The hind paws of animals from the control and IB-MECA treated groups were dissected and after protein extraction the samples were subjected for WB analysis. Modulation in the A3AR itself was noted upon treatment with IB-MECA, demonstrating that activation of the receptor and its subsequent degradation took place (FIG. 10A). The activation of the A3AR led to a 40% decrease in the RANKL protein expression level in paw extracts derived from IB-MECA treated AIA rats. Another signaling pathway which is down-regulated upon IB-MECA treatment is the PI3K-PKB/AKT, as was noted in FIG. 10C. A reduction in the protein expression level of PI3K and PKB/AKT took place in the IB-MECA treated group, in comparison to that of the control, followed by down-regulation in the level of the PKB/AKT down-stream kinase, IKK. As a result of the decrease in the level of IKK and RANKL, a reduced level of NF-κB was observed in the IB-MECA treated animals. This Chain of events led a 50% reduction in the expression level of TNF-α upon IB-MECA treatment.

1. A method for the treatment of accelerated bone resorption in a mammal subject, the method comprises administering to said subject in need of said treatment an amount of an A₃ adenosine receptor agonist (A₃AR agonist), the amount being effective to inhibit bone resorption.

2. The method of claim 1, wherein said mammal is a human subject.

3. The method of claim 1, for the treatment of inflammation induced bone resorption.

4. The method of claim 3, for the treatment of bone resorption induced by inflammatory arthritis.

5. The method of claim 1, wherein said treatment comprises oral administration of A₃AR agonist to said subject in need.

6. The method of claim 5, wherein said treatment comprises administration of A₃RA agonist to said subject once or twice daily.

7. The method of claim 1, wherein said A₃AR agonist is a compound within the scope of the general formula (I): 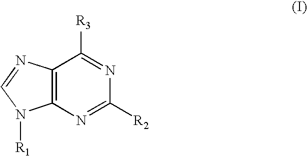
wherein,
R₁ represents an alkyl, hydroxyalkyl, carboxyalkyl or cyanoalkyl or a group of the following general formula (II): 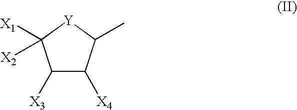
in which:
Y represents an oxygen, sulfur or CH₂;
X₁ represents H, alkyl, R^{a}R^{b}NC(═O)— or HOR^{c}—, wherein
R^{a} and R^{b} may be the same or different and are selected from the group consisting of hydrogen, alkyl, amino, haloalkyl, aminoalkyl, BOC-aminoalkyl, and cycloalkyl or are joined together to form a heterocyclic ring containing two to five carbon atoms; and
R^{c} is selected from the group consisting of alkyl, amino, lialoalkyl, aminoalkyl, BOC-aminoalkyl, and cycloalkyl;
X₂ is H, hydroxyl, alkylamino, alkylamido or hydroxyalkyl;
X₃ and X₄ represent independently hydrogen, hydroxyl, amino, amido, azido, halo, alkyl, alkoxy, carboxy, nitrilo, nitro, trifluoro, aryl, alkaryl, thio, thioester, thioether, —OCOPh, —OC(═S)OPh or both X₃ and X₄ are oxygens connected to >C═S to form a 5-membered ring, or X₂ and X₃ form the ring of formula (III): 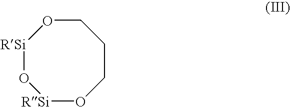
where R′ and R″ represent independently an alkyl group;
R₂ is selected from the group consisting of hydrogen, halo, alkylether, amino, hydrazido, alkylamino, alkoxy, thioalkoxy, pyridylthio, alkenyl; alkynyl, thio, and alkylthio; and
R₃ is a group of the formula —NR₄R₅ wherein
R₄ is a hydrogen atom or a group selected from alkyl, substituted alkyl or aryl-NH—C(Z)-, with Z being O, S, or NR^{a} with R^{a} having the above meanings; wherein when R₄ is hydrogen than
R₅ is selected from the group consisting of R- and S-1-phenylethyl, benzyl, phenylethyl or anilide groups unsubstituted or substituted in one or more positions with a substituent selected from the group consisting of alkyl, amino, halo, haloalkyl, nitro, hydroxyl, acetoamido, alkoxy, and sulfonic acid or a salt thereof; benzodioxanemethyl, fururyl, L-propylalanyl-aminobenzyl, β-alanylaminobenzyl, T-BOC-β-alanylaminobenzyl, phenylamino, carbamoyl, phenoxy or cycloalkyl; or R₅ is a group of the following formula: 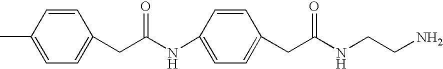
or when R₄ is an allyl or aryl-NH—C(Z)-, then, R₅ is selected from the group consisting of heteroaryl-NR^{a}-C(Z)-, heteroaryl-C(Z)-, alkaryl-N^{a}-C(Z)-, alkaryl-C(Z)-, aryl-NR-C(Z)- and aryl-C(Z)-; Z representing an oxygen, sulfor or amine; or a physiologically acceptable salt of the above compound.

8. The method of claim 1, wherein said A₃AR agonist is a nucleoside derivative of the general formula (IV): 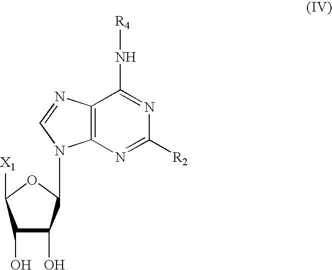
wherein X₁, R₂ and R₄ are as defined in claim 3, and physiologically acceptable salts of said compound.

9. The method of claim 1 wherein said A₃AR agonist is selected from N⁶-2-(4-aminophenyl)ethyladenosine (APNEA), N⁶-(4-amino-3-iodobenzyl) adenosine-5′-(N-methyluronamide) (AB-MECA), N⁶-(3-iodobenzyl)-adenosine-5′-N-methyluronamide (IB-MECA) and 2-chloro-N⁶-(3-iodobenzyl)-adenosine-5′-N-methyluronamide (Cl-IB-MECA).

10. The method of claim 9, wherein said A₃AR agonist is IB-MECA.

11. A pharmaceutical composition for the treatment of accelerated bone resorption, the composition comprising an amount of an A₃AR agonist, the amount being effective to inhibit bone resorption in a mammal subject.

12. The pharmaceutical composition of claim 11, in a dosage form suitable for oral administration.

13. The pharmaceutical composition of claim 11, for the treatment of inflammation induced bone resorption.

14. The pharmaceutical composition of claim 13, for the treatment of bone resorption induced by inflammatory arthritis.

15. The pharmaceutical composition of claim 11, wherein said A₃AR agonist is a compound within the scope of the general formula (I): 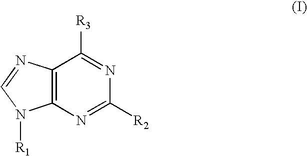
wherein,
R₁ represents an alkyl, hydroxyalkyl, carboxyalkyl or cyanoalkyl or a group of the following general formula (II): 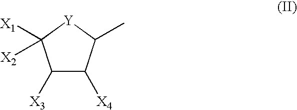
in which:
Y represents an oxygen, sulfur or CH₂;
X₁ represents H, allyl, R^{a}R^{b}NC(═O)— or HOR^{c}—, wherein
R^{a l and R}^{b} may be the same or different and are selected from the group consisting of hydrogen, alkyl, amino, haloalkyl, aminoalkyl, BOC-aminoalkyl, and cycloalkyl or are joined together to form a heterocyclic ring containing two to five carbon atoms; and
R^{c} is selected from the group consisting of alkyl, amino, haloalkyl, aminoalkyl, BOC-aminoalkyl, and cycloalkyl;
X₂ is H, hydroxyl, alkylamino, alkylamido or hydroxyalkyl;
X₃ and X₄ represent independently hydrogen, hydroxyl, amino, amido, azido, halo, allyl, alkoxy, carboxy, nitrilo, nitro, trifluoro, aryl, alkaryl, thio, thioester, thioether, —OCOPh, —OC(═S)OPh or both X₃ and X₄ are oxygens connected to >C═S to form a 5-membered ring, or X₂ and X₃ form the ring of formula (III): 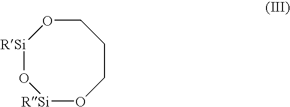
where R′ and R″ represent independently an alkyl group;
R₂ is selected from the group consisting of hydrogen, halo, alkylether, amino, hydrazido, alkylamino, alkoxy, thioalkoxy, pyridylthio, alkenyl; alkynyl, thio, and alkylthio; and
R₃ is a group of the formula —NR₄R₅ wherein
R₄ is a hydrogen atom or a group selected from alkyl, substituted alkyl or aryl-NH—C(Z)-, with Z being O, S, or NR^{a} with R^{a} having the above meanings; wherein when R₄ is hydrogen than
R₅ is selected from the group consisting of R- and S-1-phenylethyl, benzyl, phenylethyl or anilide groups unsubstituted or substituted in one or more positions with a substituent selected from the group consisting of alkyl, amino, halo, haloalkyl, nitro, hydroxyl, acetoamido, alkoxy, and sulfonic acid or a salt thereof; benzodioxanemethyl, fururyl, L-propylalanyl-aminobenzyl, β-alanylamino-benzyl, T-BOC-β-alanylaminobenzyl, phenylamino, carbamoyl, phenoxy or cycloalkyl; or R₅ is a group of the following formula: 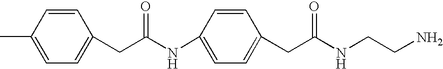
or when R₄ is an alkyl or aryl-NH—C(Z)-, then, R₅ is selected from the group consisting of heteroaryl-NR^{a}—C(Z)-, heteroaryl-C(Z)-, alkaryl-NR^{a}—C(Z)-, alkaryl-C(Z)-, aryl-NR—C(Z)- and aryl-C(Z)-; Z representing an oxygen, sulfor or amine; or a physiologically acceptable salt of the above compound.

16. The pharmaceutical composition of claim 11, wherein said A₃AR agonist is a nucleoside derivative of the general formula (IV): 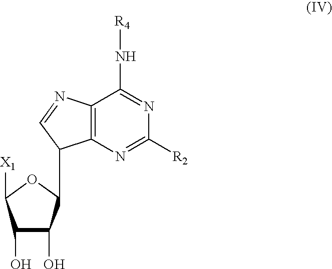
wherein X₁, R₂ and R₄ are as defined in claim 3, and physiologically acceptable salts of said compound.

17. The pharmaceutical composition of claim 11, wherein said A₃AR agonist is selected from N⁶-2-(4-aminophenyl)ethyladenosine (APNEA), N⁶-(4-amino-3-iodobenzyl) adenosine-5′-(N-methyluronamide) (AB-MECA), N⁶-(3-iodobenzyl)-adenosine-5′-N-methyluronamide (IB-MECA) and 2-chloro-N⁶-(3-iodobenzyl)-adenosine-5′-N-methyluronamide (Cl-IB-MECA).

18. The pharmaceutical composition of claim 11, wherein said A₃AR agonist is IB-MECA.

19. Use of an A₃AR agonist for the preparation of a pharmaceutical composition for the treatment of accelerated bone resorption.

20. The use of claim 19, for the preparation of a composition suitable for oral administration.

21. The use of claim 20, for the preparation of a composition the treatment of inflammation induced bone resorption.

22. The use of claim 21, wherein said composition is for the treatment of bone resorption induced by inflammatory arthritis.

23. The use of claim 19, wherein said A₃AR agonist is a compound within the scope of the general formula (I): 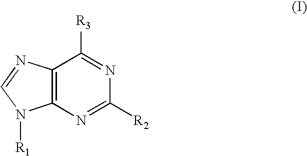
wherein,
R₁ represents an alkyl, hydroxyalkyl, carboxyalkyl or cyanoalkyl or a group of the following general formula (II): 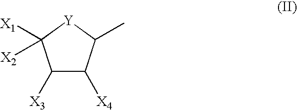
in which:
Y represents an oxygen, sulfur or CH₂;
X₁ represents H, alkyl, R^{a}R^{b}NC(═O)— or HOR^{c}—, wherein
R^{a} and R^{b} may be the same or different and are selected from the group consisting of hydrogen, alkyl, amino, haloalkyl, aminoalkyl, BOC-aminoalkyl, and cycloalkyl or are joined together to form a heterocyclic ring containing two to five carbon atoms; and
R^{c} is selected from the group consisting of alkyl, amino, haloalkyl, aminoalkyl, BOC-aminoalkyl, and cycloalkyl;
X₂ is H, hydroxyl, alkylamino, alkylamido or hydroxyalkyl;
X₃ and X₄ represent independently hydrogen, hydroxyl, amino, amido, azido, halo, alkyl, alkoxy, carboxy, nitrilo, nitro, trifluoro, aryl, alkaryl, thio, thioester, thioether, —OCOPh, —OC(═S)OPh or both X₃ and X₄ are oxygens connected to >C═S to form a 5-membered ring, or X₂ and X₃ form the ring of formula (III): 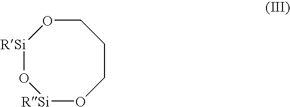
where R′ and R″ represent independently an alkyl group;
R₂ is selected from the group consisting of hydrogen, halo, alkylether, amino, hydrazido, alkylamino, alkoxy, thioalkoxy, pyridylthio, alkenyl; alkynyl, thio, and alkylthio; and
R₃ is a group of the formula —NR₄R₅ wherein
R⁴ is a hydrogen atom or a group selected from alkyl, substituted alkyl or aryl-NH—C(Z)-, with Z being O, S, or NR^{a} with R^{a} having the above meanings; wherein when R₄ is hydrogen than
R₅ is selected from the group consisting of R- and S-1-phenylethyl, benzyl, phenylethyl or anilide groups unsubstituted or substituted in one or more positions with a substituent selected from the group consisting of alkyl, amino, halo, haloallyl, nitro, hydroxyl, acetoamido, alkoxy, and sulfonic acid or a salt thereof; benzodioxanemethyl, fururyl, L-propylalanyl-aminobenzyl, β-alanylaminobenzyl, T-BOC-β-alanylminobenzyl, phenylamino, carbamoyl, phenoxy or cycloalkyl; or R₅ is a group of the following formula: 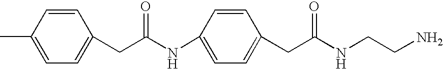
or when R₄ is an alkyl or aryl-NH—C(Z)-, then, R₅ is selected from the group consisting of heteroaryl-NR^{a}—C(Z)-, heteroaryl-C(Z)-, alkaryl-NR^{a}—C(Z)-, alkaryl-C(Z)-, aryl-NR—C(Z)- and aryl-C(Z)-; Z representing an oxygen, sulfor or amine; or a physiologically acceptable salt of the above compound.

24. The use of claim 19, wherein said A₃AR agonist is a nucleoside derivative of the general formula (IV): 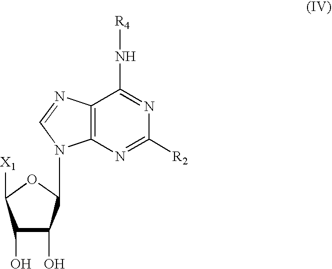
wherein X₁, R₂ and R₄ are as defined in claim 3, and physiologically acceptable salts of said compound.

25. The use of claim 19, wherein said A₃AR agonist is selected from N⁶-2-(4-aminophenyl)ethyladenosine (APNEA), N⁶-(4-amino-3-iodobenzyl) adenosine-5′-(N-methyluronamide) (AB-MECA), N⁶-(3-iodobenzyl)-adenosine-5′-N-methyluronamide (IB-MECA) and 2-chloro-N⁶-(3-iodobenzyl)-adenosine-5′-N-methyluronamide (Cl-IB-MECA).

26. The use of claim 19, wherein said A₃AR agonist is IB-MECA.
